(19) United States
(12) Patent Application Publication
(10) Pub. No.: US 2005/0031706 A1
(43) Pub. Date: Feb. 10, 2005

(54) Viscosity-stable compositions useful for treating gastrointestinal disorders

(76) Inventors: Lorraine E. Pena, Kalamazoo, MI (US); Dennis L. Huczek, Portage, MI (US); Gary D. Ewing, Kalamazoo, MI (US)

Correspondence Address: PFIZER, INC., 201 TABOR ROAD, MORRIS PLAINS, NJ, 07950, US

(21) Appl. No.: 10/843,184

(22) Filed: May 11, 2004

Related U.S. Application Data
(60) Provisional application No. 60/492,930, filed on Aug. 6, 2003.

(51) Int. Cl. A61K 33/24

(52) U.S. Cl. 424/653

(57) ABSTRACT

The present invention provides a pharmaceutical composition comprising at least one pharmaceutically acceptable bismuth-containing compound, at least one pharmaceutically acceptable non-clay-derived suspending agent, water, and a stabilizing means for reducing viscosity change upon storage of the composition. The suspension exhibits reduced change in viscosity by comparison with an otherwise similar suspension excepting the stabilizing means. Such compositions are useful in the prevention and treatment of gastrointestinal diseases and/or disorders.

This application claims the benefit of U.S. Provisional Patent Application No. 60/492,930, filed on Aug. 6, 2003, the entirety of which is hereby incorporated by reference as if fully set forth herein.

### FIELD OF THE INVENTION

The present invention relates to stable bismuth-containing pharmaceutical compositions, to methods of preparing such compositions, and to use of such compositions in treating and/or preventing gastrointestinal disorders and/or disturbances.

### BACKGROUND

Bismuth-containing pharmaceutical compositions, particularly pharmaceutical suspensions, are well known for use in treating a variety of gastrointestinal disorders including nausea, heartburn and diarrhea. Illustrative bismuth-containing suspensions currently and/or previously on the market include Kaopectate® of Pharmacia Corporation, Pepto-Bismol® of Procter & Gamble Company, several similar retail branded bismuth-containing suspensions (illustratively including those sold by Walgreen's, Rite-Aid, Spartan, and Meijer), and Pabizol with Paregoric® of Rexall. Bismuth-containing compositions are described generally in Handbook of Nonprescription Drugs, 8th Edition, American Pharmaceutical Association, Washington D.C.; 1986, pages 73-74. In addition to a bismuth-containing compound, many of these products further contain, inter alia, one or more anti-microbial preservatives, magnesium aluminum silicate and other suspending agents, colorant(s), etc.

U.S. Pat. No. 4,940,695 to Coveney discloses pharmaceutical compositions suitable for oral administration comprising pharmaceutically-acceptable bismuth-containing agents, pharmaceutically-acceptable non-ionic cellulose ether polymers, and magnesium aluminum silicate.

U.S. Pat. No. 5,013,560 to Stentz discloses microbially stable liquid pharmaceutical suspensions for oral administration comprising a bismuth-containing pharmaceutical agent, benzoic acid, sorbic acid, a suspension system preferably comprising magnesium aluminum silicate, and water, wherein the suspensions have a pH within the range of about 3.0 to about 5.5.

European Patent Application No. 0 217 440 to Gonsalves discloses pharmaceutical compositions for treatment of gastrointestinal disorders comprising 1.5% to 5% of a pharmaceutically-acceptable bismuth salt, 0.3% to 1.3% magnesium aluminum silicate, 0.5% to 0.85% xanthan gum, and water, having a defined ratio of magnesium aluminum silicate to xanthan gum.

Many currently marketed bismuth-containing suspensions exhibit the undesirable characteristic of upward pH drift which is accompanied by several potential adverse consequences. For example, most pharmaceutically acceptable anti-microbial preservatives become less effective at higher pH levels. Therefore, suspensions that exhibit an increase in pH during storage tend to more quickly reach pH levels at which one or more anti-microbial preservatives (present in the suspension) tend to be less effective or completely ineffective. Consequently, such suspensions more rapidly become susceptible to microbial contamination and have a relatively short shelf life. Additionally, many common pharmaceutical excipients, for example colorants such as indigo carmine and turmeric, are pH sensitive. Suspensions that exhibit pH drift and comprise pH-sensitive excipients tend to change appearance and/or color over time. Such changes are particularly undesirable from a commercial acceptance and product recognition standpoint.

Desirably, a bismuth-containing pharmaceutical composition should be stable upon storage for a reasonable length of time, for example 6 months. Stability, as used herein, means retention of (a) effectiveness in treating a variety of gastrointestinal disorders, (b) anti-microbial (i.e. antibacterial and anti-fungal) activity, and (c) other properties affecting palatability. Palatability, as used herein, includes properties directly affecting taste (i.e. sweetness, sourness, bitterness, and saltiness) as well as certain indirect properties (i.e. those affecting visual, aromatic, and especially viscosity profile). When the user notes changes in palatability, such changes can be perceived as being disagreeable and may cause the user to discard the suspension, regardless of whether the composition actually remains effective.

If a bismuth-containing pharmaceutical composition could be prepared which exhibits enhanced stability, in particular, improved resistance to viscosity change, a significant advance in bismuth-containing compositions would result.

### BRIEF SUMMARY OF THE INVENTION

There is now provided in the present invention an orally deliverable pharmaceutical composition in the form of a suspension comprising at least one pharmaceutically acceptable bismuth-containing agent, at least one pharmaceutically acceptable non-clay-derived suspending agent, water, and at least one stabilizing means for reducing viscosity change during storage of the composition.

The at least one stabilizing means of the instant invention can be selected from buffering agents, hydronium ions at a concentration effective to achieve a pH of the composition of about 4.0 to about 5.2, electrolytes, and salicylate agents.

Desirably, the at least one stabilizing means is present in a total amount effective to prevent more than about a 50% change in viscosity during a 5 week storage period at 50° C., the storage period being initiated within a reasonably short time after preparation of the composition, and viscosity change being expressed as change from the viscosity observed at the start of the storage period.

In one embodiment, the at least one stabilizing means of the instant composition is present at an amount effective to prevent more than about a 10% decrease in viscosity during an about 3 month storage period at 24° C., the storage period being initiated within a reasonably short time after preparation of the composition.

In another embodiment, the viscosity stabilizing means comprises a buffering agent, an electrolyte, a pH of the composition of about 4.7 to about 5.1, and a salicylate.

### BRIEF DESCRIPTION OF THE DRAWING(S)

FIG. 1 is a graph showing the effect of pH on viscosity of a formulation stored at 50° C. as described in Example 1.

FIG. 2 is a graph showing the effect of sodium salicylate on viscosity of a formulation stored at 50° C. as described in Example 2.

FIG. 3 is a graph showing the effect of pH on viscosity of a low bismuth formulation stored at 50° C. as described in Example 3.

FIG. 4 is a graph showing the effect of pH on viscosity of a low bismuth formulation stored at 50° C. as described in Example 4.

FIG. 5 is a graph showing the effect of pH on viscosity of a low bismuth formulation stored at room temperature as described in Example 4.

FIG. 6 is a graph showing the effect of pH on viscosity of a high bismuth formulation stored at 50° C. as described in Example 5.

FIG. 7 is a graph showing the effect of pH on viscosity of a high bismuth formulation stored at room temperature as described in Example 5.

FIG. 8 is a graph showing the effect of sodium chloride concentration on viscosity of a low bismuth formulation stored at 50° C. as described in Example 6.

FIG. 9 is a graph showing the effect of sodium chloride concentration on viscosity of a low bismuth formulation stored at room temperature as described in Example 6.

FIG. 10 is a graph showing the effect of sodium chloride concentration on viscosity of a high bismuth formulation stored at 50° C. as described in Example 7.

FIG. 11 is a graph showing the effect of sodium chloride concentration on viscosity of a high bismuth formulation stored at room temperature as described in Example 7.

FIG. 12 is a graph showing the effect of different buffer systems on viscosity of a low bismuth formulation stored at 50° C. as described in Example 9.

FIG. 13 is a graph showing the effect of different buffer systems on viscosity of a low bismuth formulation stored at room temperature as described in Example 9.

FIG. 14 is a graph showing the effect of different buffering systems on viscosity of a low bismuth formulation stored at 50° C. as described in Example 10.

FIG. 15 is a graph showing the effect of different buffering systems on viscosity of a low bismuth formulation stored at room temperature as described in Example 10.

FIG. 16 is a summary graph showing the effect of various conditions on viscosity of a low bismuth formulation stored at 50° C. as described in Example 11.

FIG. 17 is a summary graph showing the effect of various conditions on viscosity of a low bismuth formulation stored at room temperature as described in Example 11.

### DETAILED DESCRIPTION OF THE INVENTION

Viscosity Stabilizer

The instant composition comprises at least one pharmaceutically acceptable bismuth compound, at least one pharmaceutically acceptable non-clay-derived suspending agent, water, and at least one stabilizing means for reducing viscosity change upon storage of the composition. The at least one stabilizing means can be selected from the group consisting of buffers, hydronium ions, electrolytes, salicylates, and combinations thereof. The term “reducing viscosity change,” as used herein, means the process of preventing or reducing a change in viscosity that would otherwise occur during storage of the instant composition when compared to the composition viscosity determined at reasonably short time after preparation of the composition, absent the stabilizing means. While one skilled in the art is able to readily develop assays to quantify stabilizing means, two Viscosity Stability Tests are described below. Such tests can be used to select useful stabilizing means and amounts thereby.

Buffers

In one embodiment of the instant invention, buffers provide a stabilizing means for reducing viscosity change of the formulation. Without being bound by theory, the stabilizing means reduces viscosity, at least in part, by providing pH stability. Such buffers also have the benefit of (a) preventing unpleasant change in taste; (b) preserving effectiveness of antimicrobial agents; and (c) providing electrolyte.

While any pharmaceutically acceptable buffer may be used in the present invention, preferred buffers are selected from the group consisting of acetates, sorbates, lactates, malates, citrates, tartrates, and phosphates. More preferred buffers are selected from the group consisting of citrates (especially sodium citrate) and phosphates (especially potassium phosphate and/or sodium phosphate). An even more preferred buffer is citrate buffer.

Concentrations of buffers useful in the formulation of the instant invention can be determined by one skilled in the art based upon pH desired, pKa of the buffer used, and considerations of other components of the formulation (for example, anions and cations present). Moreover, it has been discovered herein that the effectiveness of certain preferred buffers as a stabilizing means are, in part, distinct from the buffer's direct effect on pH stability. Accordingly, concentration of buffers useful in the instant invention can be readily determined by performance in a Viscosity Stability Test as described below. For example, the aforementioned buffers have been found to be useful at concentrations of about 5 to about 100 mM, preferably about 10 to about 80 mM, and more preferably about 20 to about 65 mM, for example about 50 mM. In a preferred embodiment, the buffer is citrate adjusted to about pH 5.0 by combining 0.252% (w/v) citric acid and 1.118% (w/v) sodium citrate. The term “mM”, as used herein means millimoles per liter of the composition.

Hydronium Ions

In another embodiment of the instant invention, the at least one stabilizing means comprises hydronium ions, preferably at a concentration effective to achieve a pH of the composition of about 4.0 to about 5.2, more preferably about 4.5 to about 5.2, and more preferably still about 4.7 to about 5.2. A highly preferred initial pH is in the range of about 4.7 to about 5.1. At a pH greater than 5.2, loss of effectiveness of preservatives of the instant invention can sometimes be observed.

While any pharmaceutical source of hydronium or hydroxide ions may be used in the instant formulation to adjust the hydronium ion concentration, preferred sources are HCl and NaOH.

Electrolytes

In another embodiment, electrolytes can provide a stabilizing means for reducing viscosity change of the instant formulation. While any pharmaceutically acceptable electrolyte may be used, preferred electrolytes include NaCl, KCl, CaCl₂, and potassium sorbate. Preferably, electrolytes are present in a total electrolyte concentration of about 10 to about 200 mM, more preferably about 20 to about 150 mM, more preferably still, about 20 mM to about 100 mM, for example about 50 mM.

Salicylates.

In one embodiment, the at least one stabilizing means for reducing viscosity change of the instant composition comprises a pharmaceutically acceptable salicylate agent. While any pharmaceutically acceptable salicylate can be used, preferred salicylate agents are salicylic acid, and salicylate salts (especially sodium salicylate).

A salicylate agent, as a stabilizing means, is to be distinguished from bismuth subsalicylate that can be one embodiment of a bismuth-containing agent. Accordingly, the term “salicylate agent” is not meant to embrace bismuth subsalicylate. However, it should be noted that a salicylate agent can cooperate with bismuth subsalicylate to achieve a viscosity-stable composition of the present invention. As used herein, a viscosity-stable composition means a composition of the present invention that has reduced viscosity change during storage of the composition.

Salicylate agents of the present invention can optionally be present at a concentration of about 0.5 to about 50 mg/ml (salicylate milliequivalents) of the composition. Moreover, one skilled in the art will consider ototoxicity that can sometimes result from elevated serum levels of salicylates when preparing compositions of the present invention.

By way of example, in some circumstances, the total salicylate content of the instant invention can be contributed by bismuth subsalicylate, when present, and by a salicylate agent. Accordingly, the amount of salicylate agent to be added to the composition to stabilize viscosity must take into account such bismuth subsalicylate content.

Suspending Agents

The at least one pharmaceutically acceptable non-clay-derived suspending agent of the present invention can be inorganic or organic, polymeric or non-polymeric, and/or cellulosic or non-cellulosic. Non-limiting examples of suitable cellulosic polymers include methylcellulose, hydroxypropylmethylcellulose, hydroxybutylmethylcellulose, hydroxyethylmethylcellulose, ethylhydroxyethylcellulose, hydroxyethylcellulose, hydroxypropylcellulose, carboxymethylcellulose sodium, microcrystalline cellulose, a combination of carboxymethylcellulose sodium and microcrystalline cellulose (e.g. Avicel® RC-591 of FMC Corp.), and mixtures thereof. Preferably, the suspending agent is a non cellulosic polymer, by example, guar, propylene glycol alginate, locust bean gum, and xanthan gum. More preferably, the suspending agent comprises substantially no amount of a clay-derived suspending agent. More preferably still, the suspending agent is xanthan gum.

Bismuth Agents.

The at least one pharmaceutically acceptable bismuth-containing compound of the present invention can be any pharmaceutically acceptable bismuth-containing compound, illustratively bismuth aluminate, bismuth subcarbonate, bismuth subcitrate, bismuth nitrate, bismuth citrate, tripotassium dicitrato bismuthate, bismuth subgallate, bismuth subnitrate, bismuth tartrate, bismuth subsalicylate and mixtures thereof. Preferably, the bismuth-containing compound is in the form of a salt, more preferably bismuth subsalicylate.

Compositions of the present invention typically comprise at least one pharmaceutically acceptable bismuth-containing compound in a total amount of about 0.1 to about 500 mg/ml of the composition, preferably about 0.5 to about 250, more preferably about 1 to about 100, and still more preferably about 5 to about 50 mg/ml.

Preservatives

In a preferred embodiment, a composition of the invention comprises at least one pharmaceutically acceptable preservative agent comprising an anti-microbial activity. Non-limiting examples of the at least one preservative agent include butylparaben, editic acid, ethylparaben, glycerol, methylparaben, potassium sorbate, propionic acid, propylene glycol, propylparaben, salicylic acid, sorbic acid, sodium benzoate, sodium propionate, sodium salicylate, etc. Preferred preservative agents include sorbic acid, benzoic acid, methylparaben, salicylic acid, and salts thereof. In a more preferred embodiment, instant compositions comprise a preservative agent comprising a compound with antibacterial activity and a compound with antifungal activity. In such a preservative agent, the antibacterial activity and the antifungal activity may be in the same or in different compounds. More preferred still, the preservative agent retains substantial antimicrobial activity during storage of the composition for six months at room temperature when compared to the activity determined a reasonably short time after preparation of the composition. By way of example, a composition that meets the applicable regulatory standards during storage is considered to have the composition's antimicrobial activity “substantially maintained”.

If desired, one or more pharmaceutically acceptable anti-microbial preservatives are present in a composition of the invention in a total amount, by weight, of about 0.01% to about 10%, preferably about 0.01% to about 5%, and more preferably about 0.01% to about 2.5%.

Other Formulation Components

Compositions of the invention optionally comprise at least one anti-foaming agent. Without being bound by theory, it is believed that an anti-foaming agent present in a composition of the invention can reduce intestinal gas experienced by a subject ingesting such a composition and/or limit foaming during preparation and/or processing of a composition of the invention. Silicone-based polymers are preferred antifoaming agents. Non-limiting examples of suitable anti-foaming agents include polydimethylsiloxane (e.g. simethicone USP), 7-9245 30% simethicone emulsion of Dow Corning, Sigma Antifoam A Concentrate, and dimethicone (e.g. FG-10 anti-foam emulsion of Dow Corning). If desired, at least one anti-foaming agent is present in a composition of the invention in a total amount, by weight, of about 0.0001% to about 5%, preferably about 0.0005% to about 4%, and more preferably about 0.001% to about 2.5%.

Compositions of the invention can comprise any additional pharmaceutically acceptable excipients. The term “excipient” herein means any substance, not itself a therapeutic agent, used as a carrier or vehicle for delivery of a therapeutic agent to a subject or added to a pharmaceutical composition to improve its handling, storage, consistency, flow properties, appearance, disintegration, dispersion, dissolution, release or organoleptic properties or to permit or facilitate formation of a dose unit of the composition into a discrete article such as a capsule suitable for oral administration. Excipients can include, by way of illustration and not limitation, diluents, buffers, substances added to mask or counteract a disagreeable taste or odor, flavors, dyes, preservatives, fragrances, and substances added to improve appearance of the composition.

Viscosity Tests

Two Viscosity Stability Tests are used herein to quantify change in viscosity upon storage of the instant composition. One test was used to determine viscosity change upon storage at 50° C. Formulations were placed in a 50° C. oven within a reasonably short time after preparation of the composition. The term “within a reasonably short time after preparation of the composition” means within a period such that substantial change in viscosity was unlikely to have yet occurred, for example within one week, dependent upon storage conditions during that period.

Viscosity was monitored periodically using a Brookfield DV-II digital viscometer using spindle #34 at 12 rpm with the small volume adapter. The pH measurements were made with a Beckman Ø45 pH meter.

Similarly, viscosity change that may have occurred at room temperature was monitored as described above (except that storage was conducted at room temperature).

Variations in the precise details of these two viscosity tests are contemplated herein and such variations do not remove them from the scope of this invention.

### EXAMPLES

### Example 1

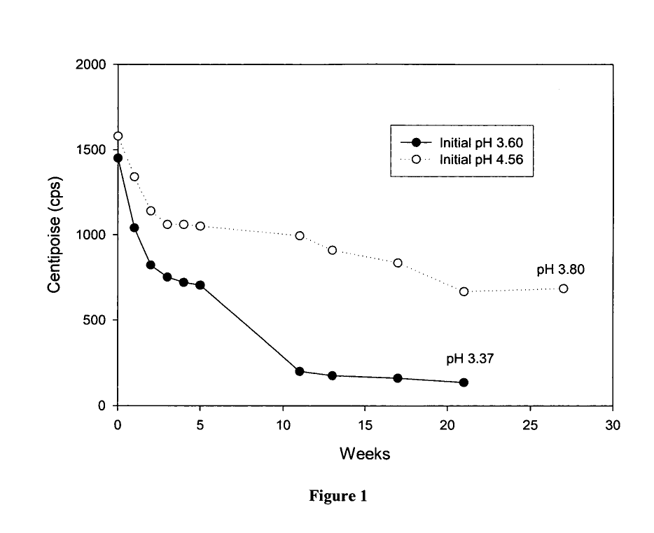

The effect of initial pH on viscosity change following storage at 50° C. at times indicated was examined on an aqueous suspension containing 0.5% xanthan gum, 17.5 mg/ml of bismuth subsalicylate (48 mM) and 0.1% sorbic acid. Viscosity change at 50° C. was monitored for the aqueous suspension at an initial pH of 3.60 (intrinsic pH) and for the aqueous suspension wherein the pH was adjusted to 4.56 with NaOH. As shown in FIG. 1, the results show that loss of viscosity was less at the higher initial pH (i.e. viscosity was better maintained). As used herein, “initial pH” means the pH observed at the start of the experiment.

### Example 2

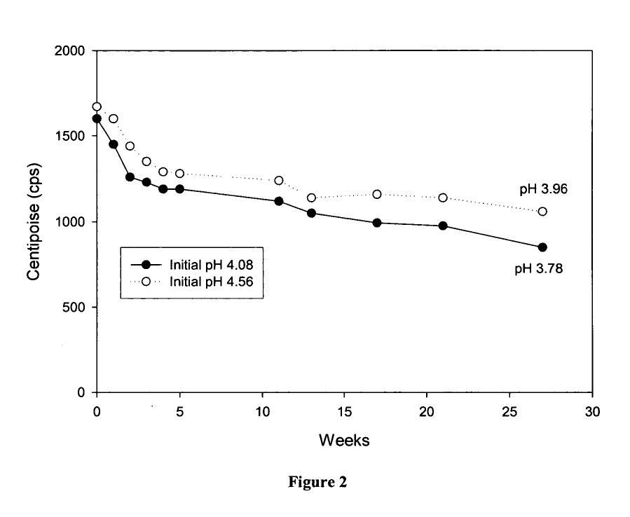

The effect on viscosity of adding 2.32 mg/ml sodium salicylate (14.5 mM) to the aqueous suspension of Example 1 is shown in FIG. 2. The data indicate that the addition of 14.5 mM sodium salicylate to a formulation containing 48 mM bismuth subsalicylate dramatically reduced the loss of viscosity over time, notwithstanding the decrease in pH observed over the course of the experiment. Without being bound by theory, these results indicate that the viscosity stabilizing effect of added sodium salicylate was, at least in part, distinct from the effect on pH.

### Example 3

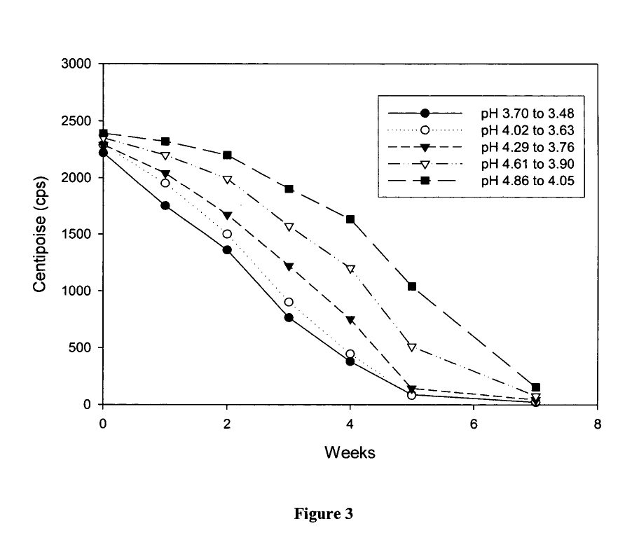

The effect of initial pH on viscosity change during storage at 50° C. of the aqueous suspension of Example 1 is shown in FIG. 3. The legend shows the initial pH and the final pH of the formulations at the end of the experiment. The data show a rank order where viscosity decreased more rapidly when the system pH was lowest. The higher the pH, the more the viscosity decrease was delayed and/or reduced (i.e. viscosity was better maintained) and the initial slopes of the lines (which represent the rate of the viscosity loss) were lower or less steep.

### Example 4

Two formulations were prepared for Examples 4 through 11, a “high” and “low” bismuth formulation. Each formulation contained suspending agents (carboxymethylcellulose sodium, microcrystalline cellulose, and xanthan gum), purified water, and sodium electrolyte (but without added NaCl). The “low bismuth” formulation additionally contained 17.5 mg/ml bismuth subsalicylate and sodium salicylate (8.7 mg/ml total salicylic acid equivalents). The “high bismuth” formulation additionally contained 35 mg/ml bismuth subsalicylate and sodium salicylate (15.7 mg/ml total salicylic acid equivalents).

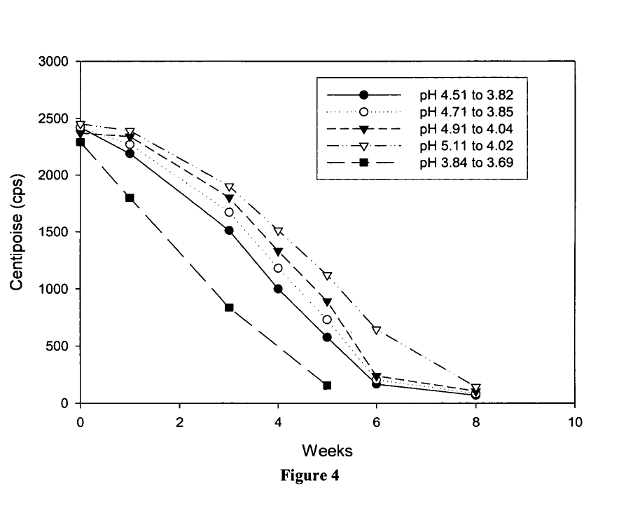

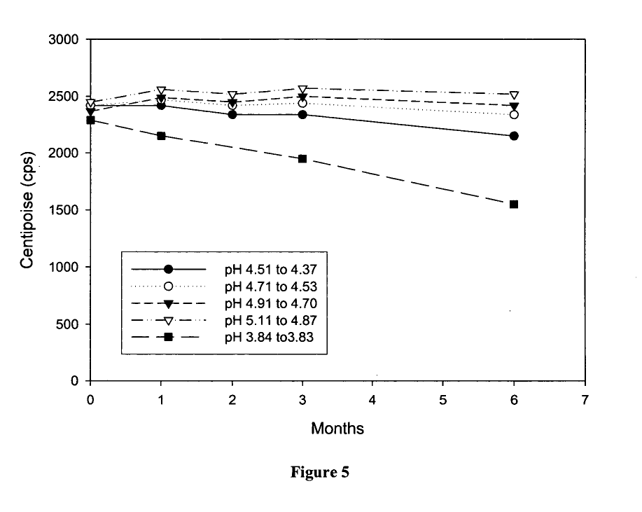

FIGS. 4 and 5 show data for the low bismuth formulation with pH adjusted to different levels. Viscosity was periodically measured while the samples were stored at 50° C. and at room temperature (˜24° C.), respectively. The legend shows the initial pH and the final pH of the formulations at the end of the experiment. Again, the higher the pH, the more the viscosity decrease was delayed and/or reduced and the initial slopes of the lines (which represent the rate of the viscosity decrease) were lower or less steep. Moreover, loss of viscosity at room temperature storage is markedly diminished.

### Example 5

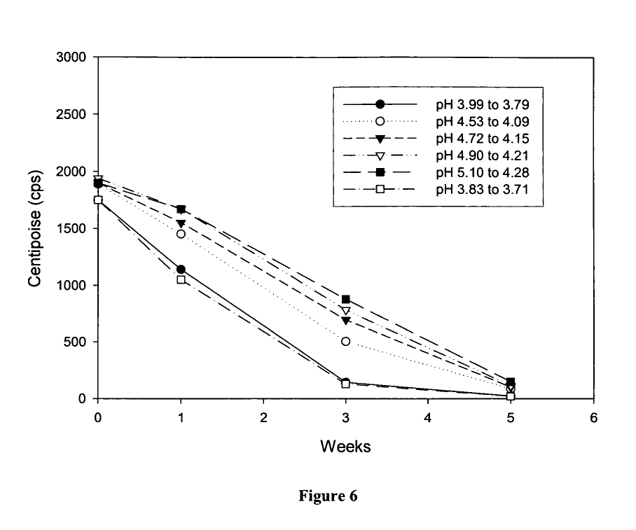

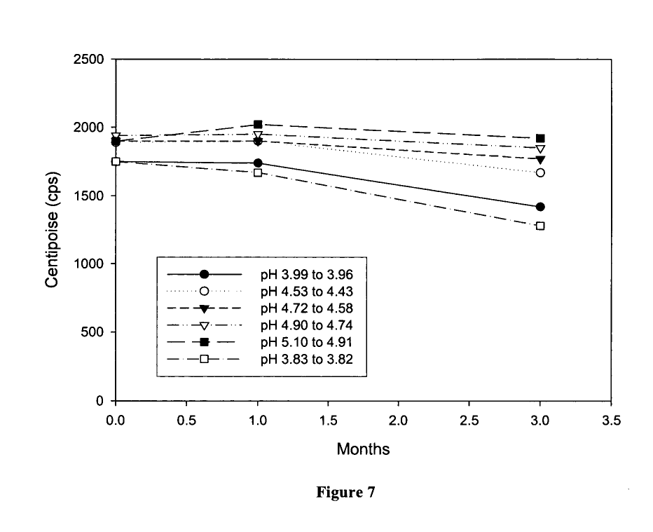

FIGS. 6 and 7 show data for the high bismuth formulations with pH adjusted to different levels Results of this study were similar to the study of Example 4. Viscosity was periodically measured while the samples were stored at 50° C. and at room temperature (˜24° C.), respectively. The legend shows the pHs of the formulations at the beginning (“initial pH”) and at the end of the experiment. Again, the higher the initial pH, the more the viscosity was maintained and the initial slopes were lower or less steep.

### Example 6

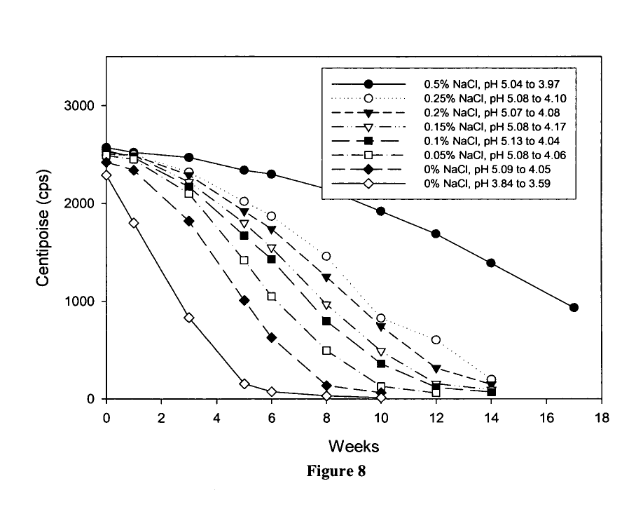

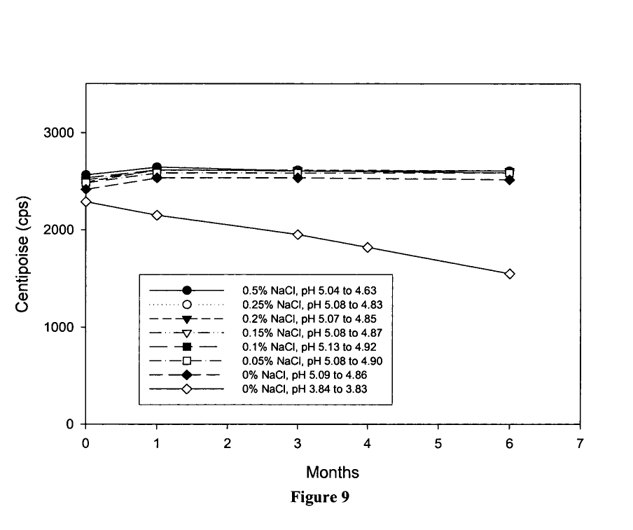

The effect of different amounts of sodium chloride added to low bismuth formulations (adjusted to an initial pH of 5.1) on viscosity during storage at 50° C. and at room temperature is shown in FIGS. 8 and 9 (respectively). The data indicate that the higher the salt content, the more viscosity loss can be delayed and/or reduced during 50° C. storage. The loss of viscosity was greatly diminished at room temperature (FIG. 9).

### Example 7

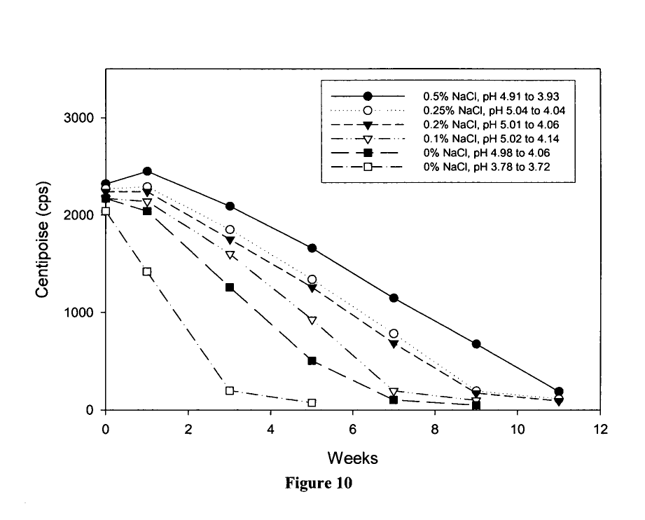

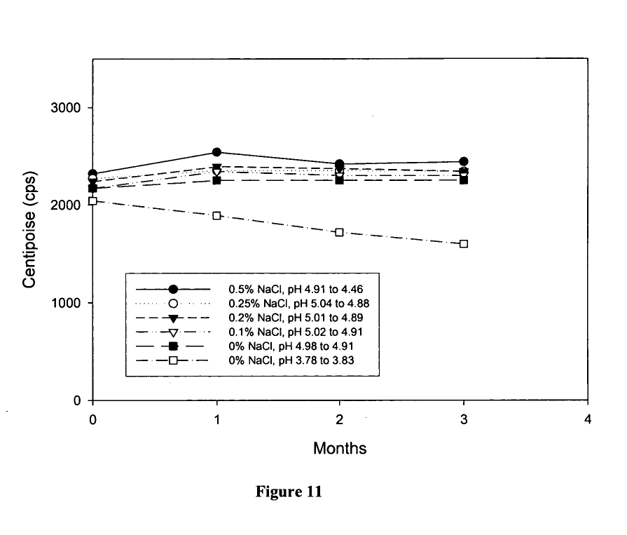

The effect of different amounts of sodium chloride added to high bismuth formulations (adjusted to an initial pH of 5.1) on viscosity during storage at 50° C. and room temperature is shown in FIGS. 10 and 11 (respectively). The results were similar to those of Example 6, however the change in viscosity was more marked.

### Example 8

Studies similar to Examples 6 and 7 were performed except that KCl or CaCl₂ were substituted for NaCl. In data not shown, results were similar to those shown in FIGS. 8-11. KCl and CaCl₂ added to the aqueous suspension prevented viscosity change.

### Example 9

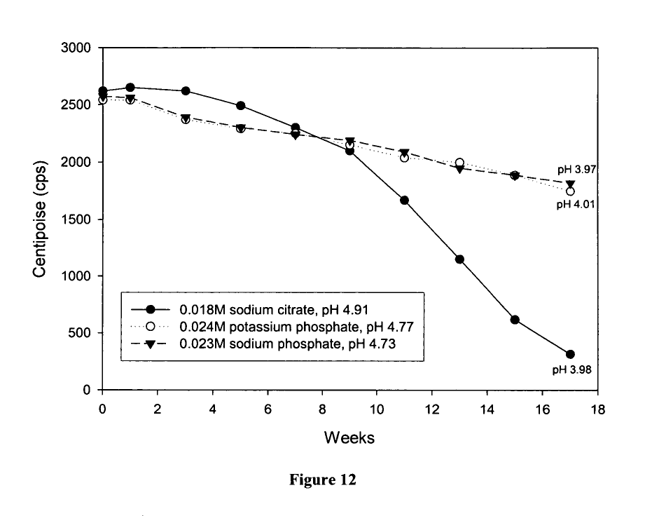

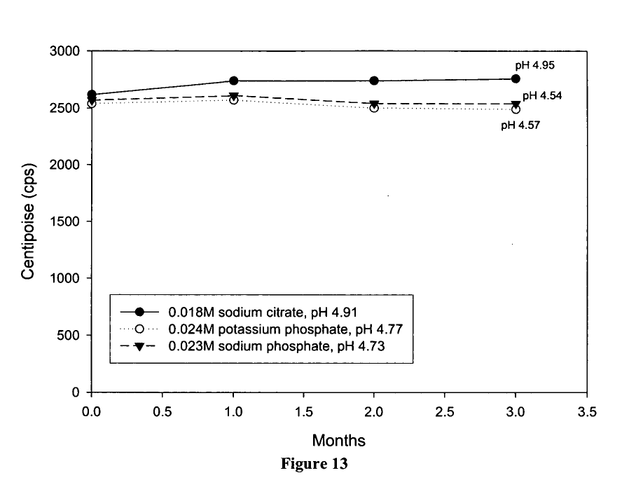

The effect of different buffer systems in the low bismuth formulation on viscosity during storage at 50° C. and room temperature is shown in FIGS. 12 and 13 (respectively). Three different buffer systems were used: sodium citrate at a concentration of 18 mM (pH 4.91), potassium phosphate at a concentration of 24 mM (pH 4.77), and sodium phosphate at a concentration of 23 mM (pH 4.73). Initial pHs are shown in the figure legend and final pHs are shown on the graph at the final time points. At room temperature (FIG. 13) over the period of data collected, each buffer system showed favorable resistance to viscosity change.

### Example 10

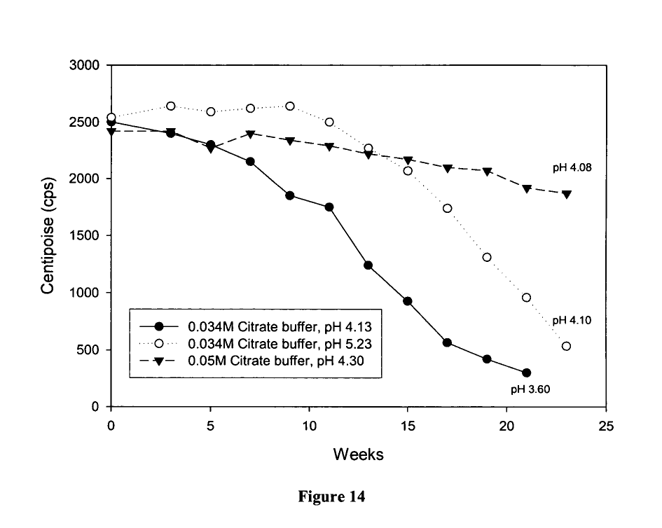

The effect of different amounts of citrate buffer at different pHs on a low bismuth formulation was examined. Citric acid and sodium citrate were added to provide electrolyte and pH adjustment. Specifically, citrate and citric acid were added to achieve 34 mM total citrate at pH 4.13 or 5.23 or to achieve 50 mM total citrate at pH 4.3. FIG. 14 shows the results of viscosity upon storage at 50° C. The buffer concentration of 50 mM citrate buffer at pH 4.30 inhibited the viscosity loss at 50° C. that was observed with the buffer concentration at 34 mM and initial pH at 4.13 or 5.23. The 50 mM citrate buffer system at pH 4.30 also resulted in less of a pH decrease than that observed with the 34 mM systems. Nevertheless, each of the buffer systems provided favorable reduction in viscosity change over the low bismuth formulation with no additions.

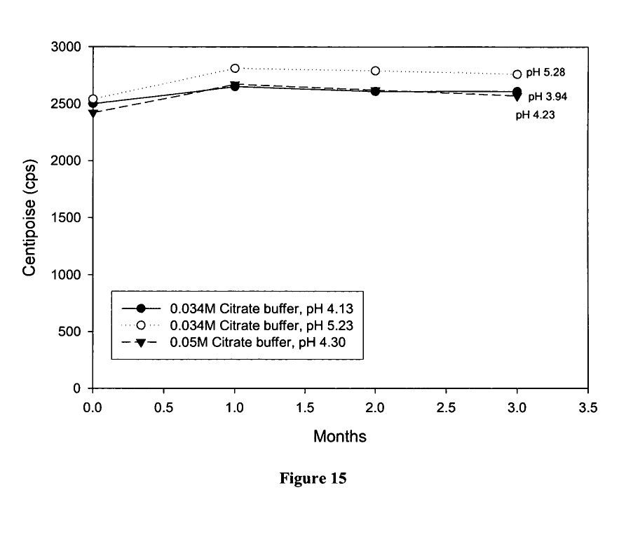

FIG. 15 shows the results of room temperature storage. Through 3 months of storage, the data indicated no loss of viscosity for any of the systems. The systems also experienced minimal pH change at room temperature conditions through 3 months.

### Example 11

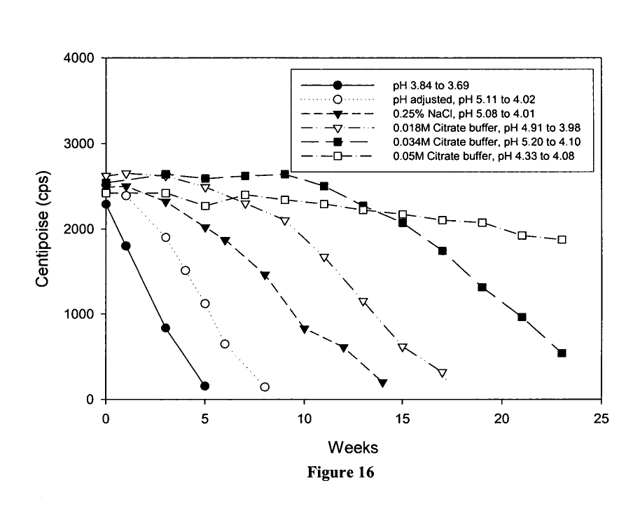

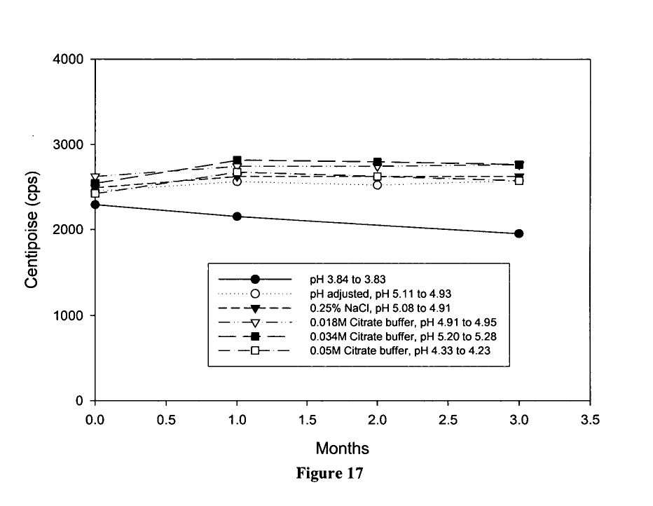

FIGS. 16 and 17 show summary data of low bismuth studies reported herein at 50° C. and room temperature, respectively. Of the formulations shown, 50 mM citrate at an initial pH of 4.33 showed the least drop in viscosity.

1. An orally deliverable pharmaceutical composition comprising
(a) at least one pharmaceutically acceptable bismuth-containing agent;
(b) at least one pharmaceutically acceptable non-clay-derived suspending agent;
c) at least one stabilizing means for reducing viscosity change during storage of the composition;
(d) and water;
wherein the composition is in the form of a suspension.

2. The composition of claim 1 wherein the at least one stabilizing means is present at a concentration effective to prevent more than about a 50% viscosity change during a 5 week storage period at 50° C., the storage period being initiated within a reasonably short time after preparation of the composition.

3. The composition of claim 1 wherein the at least one stabilizing means is present at a concentration effective to prevent more than about a 50% viscosity change during a 4 week storage period at 50° C., the storage period being initiated within a reasonably short time after preparation of the composition.

4. The composition of claim 1 wherein the at least one pharmaceutically acceptable bismuth-containing agent is present in the composition at more than about 55 mM, wherein the at least one stabilizing means is present at a concentration effective to prevent more than about a 75% viscosity change during a 3 week storage period at 50° C., the storage period being initiated within a reasonably short time after preparation of the composition.

5. The composition of claim 1 wherein the at least one stabilizing means is present at a concentration to prevent more than about 10% decrease in viscosity during a 3 month storage period at 24° C., the storage period being initiated within a reasonably short time after preparation of the composition.

6. The composition of claim 1 wherein the at least one stabilizing means is selected from the group consisting of buffering agents, hydronium ions at a concentration effective to achieve a pH of the composition of about 4.0 to about 5.2, electrolytes, and salicylate agents.

7. The composition of claim 1 wherein the at least one stabilizing means comprises at least one buffering agent.

8. The composition of claim 7 wherein the at least one buffering agent is selected from the group consisting of acetates, sorbates, lactates, malates, tartrates, citrates, and phosphates.

9. The composition of claim 7 wherein the at least one buffering agent comprises citrate.

10. The composition of claim 9 wherein the citrate comprises citric acid and citrate salt wherein the citrate is present in an amount effective to achieve a pH of about 4.5 to about 5.2.

11. The composition of claim 7 wherein the at least one buffering agent is present in the composition in a total concentration of about 5 to about 100 mM.

12. The composition of claim 7 wherein the at least one buffering agent is present in the composition in a total concentration of about 20 to about 65 mM.

13. The composition of claim 1 wherein the at least one stabilizing means comprises at least one electrolyte.

14. The composition of claim 13 wherein the at least one electrolyte is selected from the group consisting of NaCl, KCl, CaCl₂, and potassium sorbate.

15. The composition of claim 13 wherein the at least one electrolyte is present in the composition in a total concentration of about 10 to about 200 mM.

16. The composition of claim 13 wherein the at least one electrolyte is present in the composition in a total concentration of about 20 to about 100 mM.

17. The composition of claim 13 wherein the at least one electrolyte is present in the composition in a total concentration of about 30 to about 90 mM.

18. The composition of claim 1 wherein the at least one stabilizing means comprises hydronium ions at a concentration effective to achieve a pH of the composition of about 4.0 to about 5.2.

19. The composition of claim 18 wherein the hydronium ions are present at a concentration effective to achieve a pH of the composition of about 4.3 to about 5.2.

20. The composition of claim 18 wherein the hydronium ions are present at a concentration effective to achieve a pH of the composition of about 4.5 to about 5.2.

21. The composition of claim 18 wherein the hydronium ions are present at a concentration effective to achieve a pH of the composition of about 4.7 to about 5.1.

22 The composition of claim 1 wherein the at least one stabilizing means consists of at least one salicylate agent.

23. The composition of claim 22 wherein the at least one salicylate agent is present in an amount of about 0.5 to about 50 mg/ml of the composition.

24. The composition of claim 1 wherein the at least one stabilizing means comprises
(a) a buffering agent selected from the group consisting of acetates, sorbates, lactates, malates, tartrates, citrates, and phosphates, wherein the buffering agent is present in the composition in a total concentration of about 5 to about 100 mM.
(b) an electrolyte selected from the group consisting of NaCl, KCl, CaCl₂, and potassium sorbate, wherein the electrolyte is present in the composition in a total concentration of about 10 to about 200 mM.
(c) hydronium ions present at a concentration effective to achieve a pH of the composition of about 4.7 to about 5.1, and
(d) at least one salicylate agent.

25. The composition of claim 1 wherein the suspending agent comprises substantially no amount of a clay-derived suspending agent.

26. The composition of claim 25 wherein the suspending agent is a polymer.

27. The composition of claim 26 wherein the suspending agent is cellulosic.

28. The composition of claim 26 wherein the suspending agent is selected from the group consisting of guar gum, propylene glycol alginate, locust bean gum, and xanthan gum.

29. The composition of claim 1 wherein the bismuth-containing agent is selected from the group consisting of bismuth aluminate, bismuth subcarbonate, bismuth subcitrate, bismuth nitrate, bismuth citrate, tripotassium dicitrato bismuthate, bismuth subgallate, bismuth subnitrate, bismuth tartrate, and bismuth subsalicylate.

30. The composition of claim 1 wherein the bismuth-containing agent is present in a total agent amount of about 0.1 to about 500 mg/ml of the composition.

31. The composition of claim 1 wherein the bismuth-containing agent is present in a total agent amount of about 5 to about 50 mg/ml of the composition.

32. The composition of claim 1 further comprising at least one preservative agent comprising an antimicrobial compound having antimicrobial activity.

33. The composition of claim 32 wherein the at least one preservative agent substantially retains said antimicrobial activity during a 6 month storage period of the composition at room temperature.
